(19) United States
(12) Patent Application Publication
(10) Pub. No.: US 2025/0366470 A1
(43) Pub. Date: Dec. 4, 2025

(54) ANTIMICROBIAL MONOMER COATINGS AND METHODS OF MAKING AND USING SAME

(71) Applicant: Fairleigh Dickinson University, Teaneck, NJ (US)

(72) Inventor: Christian Traba, Bayonne, NJ (US)

(73) Assignee: Fairleigh Dickinson University, Teaneck, NJ (US)

(21) Appl. No.: 18/862,076

(22) PCT Filed: Apr. 13, 2023
(86) PCT No.: PCT/US2023/018423

§ 371 Date: Oct. 31, 2024

Related U.S. Application Data
(60) Provisional application No. 63/330,919, filed on Apr. 14, 2022.

(51) Int. Cl. A01N 25/10; A01N 33/12; A01P 1/00

(52) U.S. Cl. CPC A01N 25/10; A01N 33/12; A01P 1/00

(57) ABSTRACT

The present invention relates to methods of producing antimicrobial coatings on biomaterial substrates. The methods comprise: a) applying an inert plasma directly to a biomaterial substrate for about 1-5 minutes at a discharge power of about 60-100 watts, thereby producing an activated biomaterial, b) exposing the activated biomaterial to air for about 50 to about 100 minutes to generate hydroperoxide, hydroxyl, and/or peroxide reactive centers on the activated biomaterial, and c) placing the air-exposed activated biomaterial into an ammonium monomer solution with a concentration of about 20-50% (w/v) to induce graft polymerization, thereby initiating a free-radical surface grafting at reactive centers.

### BACKGROUND OF THE INVENTION

Traditional anti-infection surfaces are loaded with antibiotics or other types of antibacterial agents. Such anti-infection surfaces have been introduced in healthcare settings with the aim of supporting existing hygiene procedures, and to help combat the increasing threat of antimicrobial resistance. However, concerns have been raised over the potential selection pressure exerted by such surfaces, which may drive the evolution and spread of antimicrobial resistance. There is a need for anti-infection surfaces which do not lead to antimicrobial resistance.

### SUMMARY OF THE INVENTION

The present invention provides antimicrobial coatings which are active against adherent bacteria and biofilms and can kill them indiscriminately, at the site of adherence, thereby enabling long-term eradication of infections of biomaterials without the use of antibiotics. Several attractive advantages of the coatings of the present invention in comparison to traditional anti-infection surfaces include (1) their broad spectrum of activity against antibiotic resistant bacteria, (2) the unlikelihood of bacterial resistance (due to the proposed mechanism of action), (3) specificity, (4) biocompatibility, and (5) stability.

In one embodiment, the present invention provides an antimicrobial coating produced by a process which comprises: a) applying an inert plasma directly to a biomaterial substrate for about 1-5 minutes at a discharge power of about 60-100 watts, thereby producing an activated biomaterial, b) exposing the activated biomaterial to air for about 50 to about 100 minutes to generate hydroperoxide, hydroxyl, and/or peroxide reactive centers on the activated biomaterial, and c) placing the air-exposed activated biomaterial into an ammonium monomer solution with a concentration of about 20-50% (w/v) to induce graft polymerization to initiate a free-radical surface grafting at reactive centers. In this manner, an antimicrobial biocoating is produced on a biomaterial substrate.

In some embodiments, the biomaterial substrate comprises polyethylene terephthalate (PET), silicon wafer, cyclic olefin copolymer (COC), polycarbonate (PC), polyetherimide (PEI), medical grade polyvinylchloride (PVC), polyethersulfone (PES), polyethylene (PE), polyetheretherketone (PEEK) and/or polypropylene (PP). In some embodiments, the ammonium monomer is selected from the group consisting of allyltrimethyl ammonium, allyltriethyl ammonium, allyltripropyl ammonium, allyltributyl ammonium, allyltripentyl ammonium, allyltrihexyl ammonium, allyltriheptyl ammonium, allyltrioctyl ammonium, allyltrinonyl ammonium and combinations thereof. In some embodiments, graft polymerization occurs at about 50° C. to about 90° C. under a nitrogen atmosphere for about 6-10 hours. In some embodiments, the inert plasma is argon plasma. In some embodiments, the polymer brush length is about 270 nm to about 470 nm. In some embodiments, the polymer graft density is about 50 to about 200 μg/cm². In some embodiments, the coating inhibits the formation of biofilms. In some embodiments, the coating inhibits gram-positive and gram-negative bacteria. In some embodiments, the coating inhibits S. aureus, S. epidermidis and E. coli bacteria.

### BRIEF DESCRIPTION OF THE DRAWINGS

FIG. 1: A schematic diagram of the sPBB fabrication process.

FIG. 2: ATR-FTIR spectra (A), with corresponding AFM images (B), and Live/Dead confocal microscopy images of adherent S. aureus bacteria cultured for 18 hours (C), on the surfaces of plasma untreated (1), plasma treated (2), and sPBBs (3). Scan distance is 5 μm on AFM and the scale bar for confocal microscopy is 20 μm.

FIG. 3: sPBBs, in comparison to other biomaterials, caused the inactivation of methicillin-resistant S. aureus, S. epidermidis, and E. coli adherent bacteria. Bacterial cell death (A), along with corresponding SEM (X), and Live/Dead confocal microscopy (Y), images of S. epidermidis (1), and E. coli (2), planktonic bacteria cultured on biomaterials for 18 hours. sPBBs. in comparison to other plasma treated biomaterials, caused the inactivation of S. aureus, S. epidermidis, and E. coli biofilms (B). 7-day old mature preformed biofilms were seeded on different biomaterials and incubated for 7 days at 37° C. under stationary conditions. Live bacteria on the different biomaterials at the end of cultivation were quantified using the MTT assay. Viable bacterial numbers on surfaces were quantified by measuring OD₅₇₀ nm changes after exposure to the different surfaces. Data represent the mean and SD of twenty samples. Controls vs. sPBBs, P<0.04. Scale bar=1 μm for SEM images and 20 μm for confocal images.

FIG. 4: SEM (A), with corresponding Live/Dead confocal microscopy (B), images of S. aureus (1), S. epidermidis (2), and E. coli (3), biofilms cultured on different biomaterials for 7 days. Scale bar=1 μm for SEM images and 20 μm for confocal images.

FIG. 5: Counting of human fetal osteoblasts (A), with respective MTT absorbance readings (B), and Live/Dead confocal microscopy images (C), of cells grown on untreated biomaterials (1), plasma treated biomaterials (2), and sPBBs (3), in 6-well plates for 7 days. Cells grown on untreated biomaterials were taken as 100%. Data represent the mean and SD of twenty samples. Controls vs. sPBBs, P<0.03. Scale bar=50 μm.

FIG. 6: Release of ammonium polymers from sPBBs. Samples were incubated at 37° C. for 15 days. Studies were performed in: 2% fetal bovine serum (FBS) or simulated body fluid (SBF) (A), and PBS with pH ranges from 10 to 4 (B). Data represent the mean and SD of twenty samples.

### DETAILED DESCRIPTION OF THE INVENTION

The instant invention relates to ammonium monomers having specific chemical properties, and the use of these monomers to produce antimicrobial coatings. These coatings are referred to as “smart Polymer Brush Biocoatings” (i.e., sPBBs). The invention includes methods of synthesizing these monomers and methods of attaching these monomers to materials thereby creating antimicrobial coatings. For example, the coatings are capable of killing of both gram-positive and gram-negative bacteria. These coatings are antibiotic-free and are not toxic to the body.

In some embodiments, these sPBBs are used to coat biomaterials. Biomaterials are synthetic (e.g., metal or polymer) or natural materials that are suitable for introduction into living tissue especially as part of a medical device (e.g., artificial joints, artificial organs, catheters, stents, prostheses, bone/tissue replacements). Some examples of biomaterials include polyethylene terephthalate (PET), silicon wafer, cyclic olefin copolymer (COC), polycarbonate (PC), polyetherimide (PEI), medical grade polyvinylchloride (PVC), polyethersulfone (PES), polyethylene (PE), polyetheretherketone (PEEK) and polypropylene (PP).

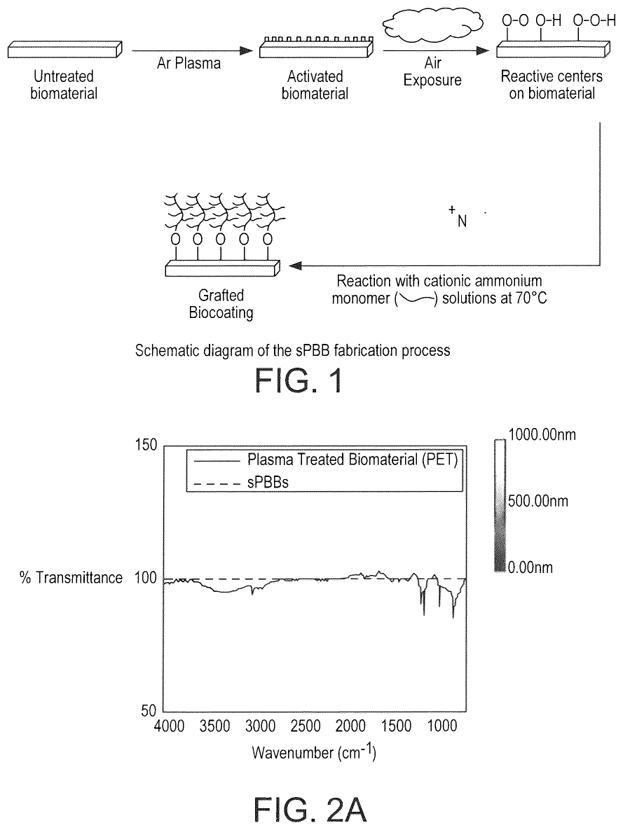

The construction of the sPBBs of the present invention are generated through a multi-step process. As depicted in FIG. 1, in the first step, an inert plasma (e.g., an argon plasma) is applied directly to a biomaterial substrate for about 1-5 minutes, typically about 3 minutes, at a discharge power of about 60-100 watts, typically about 80 watts. Preferably, the inert plasma is gentle and does not alter the bulk chemical composition of the biomaterial. These experimental conditions ensure uniform exposure to the inert plasma and activate the biomaterial creating optimized grafting density. Next, the activated biomaterial is exposed to the air for about 50 to about 100 minutes, typically about 75 minutes, to generate hydroperoxide, hydroxyl, and peroxide reactive centers.

After exposure to the air, the plasma treated biomaterial is then placed into an ammonium monomer solution with a concentration of about 20-50% (w/v), typically about 35% (w/v), to induce graft polymerization. Typically, the solution is an aqueous solution. The graft polymerization reaction is carried out at about 50° C. to about 90° C., typically about 70° C., under a nitrogen atmosphere for about 6-10 hours, typically about 8 hours, which initiates a free-radical surface grafting mechanism at reactive centers. With the plasma-initiated graft polymerization technique, a desired monomer is covalently bonded to the surface of the activated biomaterial resulting in a polymer brush layer or biocoating.

After washing (e.g., with Milli-Q water) to remove unbound ammonium polymers, sPBBs were characterized and the formation dynamics and antibiotic sensitivity of three adherent bacteria and mature biofilms on the sPBBs were studied, as well as biocompatibility, stability, and the mechanism of action.

The ammonium monomers of the solution have no inherent toxicity, antibacterial or anti-biofilm activity. Examples of ammonium monomers suitable for the present invention include: 1) allyltrimethyl ammonium, 2) allyltriethyl ammonium, 3) allyltripropyl ammonium, 4) allyltributyl ammonium, 5) allyltripentyl ammonium, 6) allyltrihexyl ammonium, 7) allyltriheptyl ammonium, 8) allyltrioctyl ammonium, 9) allyltrinonyl ammonium.

The sPBBs can carry functional groups which display chemical properties that differ from the substrate surfaces to which they are covalently bond, depending on the intended end use. For example, monomers with hydrophilic properties can make hydrophilic coatings while monomers with hydrophobic properties can make hydrophobic coatings. Examples of hydrophilic functional groups include hydroxyl groups, carbonyl groups, carboxyl groups (e.g., acrylic acid, methyl acrylate), amino groups, sulfhydryl groups, phosphate groups. Preferred groups include 1) methyl acrylic acid, 2) sorbic acid, 3) vinyl sulfonic acid. 4) cinnamic acid, 5) vinyl alcohol, 6) fumaric acid, 7) methyl acrylate. Examples of hydrophobic functional groups are alkyl groups, e.g., methyl, ethyl, triethyl, propyl groups. Thus, by varying the functional groups on the cationic ammonium monomers, biocoatings can be made which exhibit anti-fouling properties and biocompatibility.

Without wanting to be bound by a mechanism, it is believed that the physical attributes of the coatings which yield the antimicrobial properties are based on polymer graft density and brush length (amount of monomers). For example, the ammonium polymer brushes have a graft density of about 50 μg/cm² to about 200 μg/cm², more typically about 100 μg/cm² to about 150 μg/cm², and most typically about 120 μg/cm². And, the dry polymer brush length of these coatings is about 270 nm to about 470 nm, more typically about 320 nm to about 420 nm, and most typically about 370 nm. In one embodiment, the coatings possess the ammonium polymer brushes with a graft density of about 120 μg/cm² and dry polymer brush lengths of about 370 nm #: 36 nm. The coatings of the present invention enable the inhibition of the formation of biofilms.

The graft density, length and composition of the biocoatings are controlled by adjusting two different parameters: 1) exposure of the biomaterial to inert discharge gas; and 2) reaction conditions during the free-radical polymerization process. Plasma power, plasma treatment time and air exposure time affect polymer graft density. That is, the graft density increases as the plasma power, plasma treatment and/or air exposure time is increased. Monomer solution concentration and solution reaction time affect polymer brush length. That is, the brush length increases as the solution is more concentrated and/or the reaction time is increased.

(Examples of cationic monomers that are typically not suitable for the present invention are those having long alkyl chains, e.g., N,N′-bis[2-dodecyloxy-2-oxoethyl]-N,N,N′,N′-tetramethylethane-1,2-diammonium dichloride, 4,4′-(2,9-dioxadecane)bis(1-alkylpyridinium bromide), and N-cetylpyridinium chloride.)

### EXAMPLES

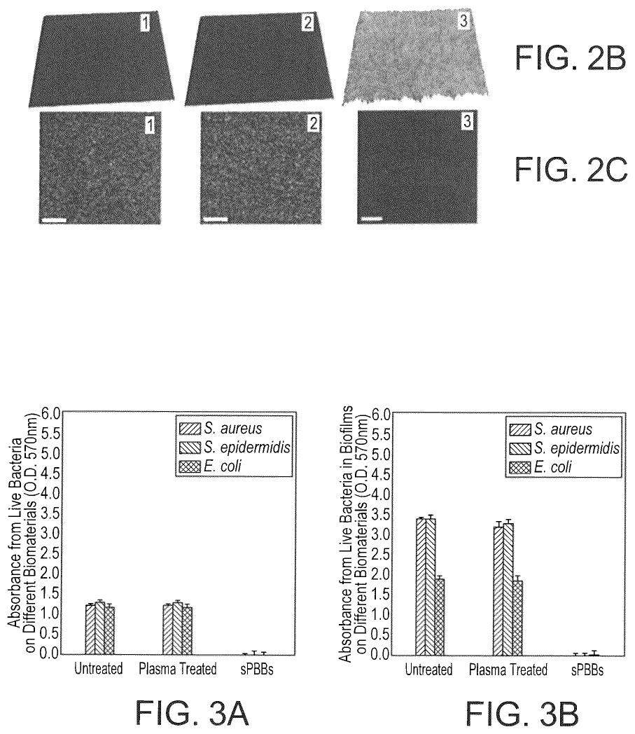

The construction of sPPBs was confirmed using Attenuated Total Reflectance Fourier Transform Infrared Spectroscopy (ATR-FTIR), Atomic Force Microscopy (AFM) and Scanning Electron Microscopy (SEM). As sPBBs were constructed, the physical and chemical properties of the surface changed significantly. When the ATR-FTIR spectrum of the plasma treated biomaterial was compared to the sPBBs, there was clear evidence of the ammonium polymers covalently bound to the PET film, demonstrated with an absorption band around 970 cm⁻¹. The peak around 2925 cm⁻¹ found on the ammonium grafted biocoating is attributed to the asymmetric stretching vibrations of the C—H bonds, stemming from the ethyl groups of the synthesized ammonium monomer (FIG. 2A).

The “grafting from” approach resulted in uniform and complete coverage and the dramatic surface topography changes after the sPBB fabrication process and were visualized using AFM (FIG. 2B). When compared to untreated silicon wafers (FIG. 2B₁) and plasma treated silicon wafers (FIG. 2B₂), the increase in thickness of the biocoatings (FIG. 2B₃) is associated with the physical properties corresponding to the grafting density and brush length of the ammonium polymers covalently bound to the surface. Atomic force microscopy images of the biocoatings depict very smooth surfaces with uniform coverage throughout the grafted biocoating (FIG. 2B₃). The grafting density for the sPBBs constructed and used throughout the study was calculated to be 120 μg/cm² according to the Orange II staining protocol. The dry polymer brush lengths of sPBBs were determined to be 370±36 nm, respectively, according to AFM images.

In this study. the formation dynamics and anti-infection sensitivity of both adherent bacteria and biofilms cultured on the sPBBs were studied. Studies revealed that desired anti-infection activity of sPBBs can be attained by optimizing polymer brush graft content and polymer brush length during the grafting procedure. The anti-bacterial activity of sPBBs was evaluated and tested in vitro by loading three different strains of bacteria (S. aureus, S. epidermidis and E. coli) onto different biomaterials and incubating the contaminated materials at 37° C. in TSBG medium overnight. All preliminary experiments are designed to model biofilm formation on catheters. Bacterial adhesion and biofilm formation on modified biomaterials were studied using three different viability assays: 1) Live/Dead kit; 2) MTT, which quantitatively measures the metabolic activity of live cells, and has an excellent correlation with colony forming unit counting (CFU) counting; and 3) CFU counting. The SYTO 9 dye in the Live/Dead kit, is a nucleic acid probe with green fluorescent color, permeates healthy cell membranes, so that cells with intact (live) cell membranes are stained green. The propidium iodide dye of the Live/Dead kit, is a nucleic acid probe with red fluorescent color, is cell membrane impermeable and thus only stains dead cells with damaged cell membranes red. Control biomaterials (untreated and plasma treated), used in FIG. 2C, provided suitable environments for the development of infections. However, at the end of overnight incubation, sPBBs demonstrated very potent anti-infection activities against S. aureus bacteria (FIG. 2C). From the results in FIG. 2C, it can be deduced that sPBBs were effective at killing adherent bacteria, thereby preventing the formation of biofilms in overnight cultures. Aside from the anti-bacterial properties of sPBBs, we also noticed a significant reduction in the numbers of adherent bacteria in comparison to control biomaterials in both SEM and confocal microscopy imaging (FIG. 2C). Experiments are addressed to test the anti-adhesive properties of sPBBs.

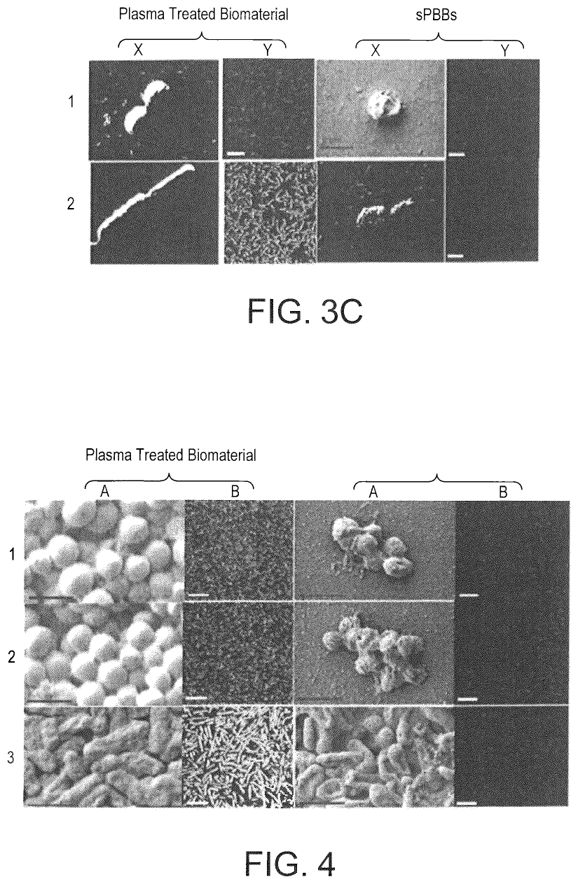

The anti-bacterial properties of sPBBs were further explored and tested against S. aureus, S. epidermidis and E. coli bacteria (FIG. 3A). The results demonstrated the same anti-bacterial activity regardless of bacterial strain. SEM and confocal microscopy images of adherent bacteria on biomaterials were taken and were useful in confirming the anti-bacterial activity of sPBBs (FIG. 3B). SEM images of control biomaterials contaminated with bacteria overnight show healthy, round and full-shaped bacteria, demonstrating the intermediate stages of biofilm formation (FIG. 3B). On the other hand, bacteria used to contaminate sPBBs depict adherent bacteria with completely damaged cell membranes (FIG. 3B). Damage of cell membranes is visualized by deformed and unsymmetrical shaped bacterial cells, with what appears to be holes or crevasses in the cell membrane (FIG. 3B). Corresponding confocal microscopy was used to further evaluate the anti-bacterial efficacy of sPBBs after exposure to bacteria for 18 hours and revealed that sPBBs caused bacterial death and subsequently, the prevention of possible infection, indicated by the red color (FIG. 3B).

As studies progressed to the inactivation of mature biofilms, sPPBs were also found to be very effective at killing mature biofilms indiscriminately as demonstrated in the MTT assay (FIG. 3C). SEM and corresponding Live/Dead confocal imaging were both used to visualize the integrity of biofilms adhered onto surfaces. Images show severe damage to the negatively-charged extracellular polymeric matrix of 7-day biofilms, as well as to the cell wall of the bacteria in biofilms after adhesion on to sPBBs, indicating cell lysis as a possible means for bacterial cell death (FIG. 4). Since the monomers used in this study lack the long alkyl functional group necessary for alkyl insertion (C12 to C16), a different antibacterial mechanism of action is proposed, similar to that of cationic antimicrobial peptides. In this proposed mechanism of action, the overall positive charge on sPBBs, which is dependent on the grafting content and length of polymer brushes, facilitates the anti-bacterial activity through electrostatic interaction between the brushes and the biofilm. In the first step, the sPBBs bind to the negatively charged biofilm, disrupting the normal functions of the biofilm, resulting in biofilm erosion. Once the architecture of biofilms is damaged, the extracellular polymeric matrix becomes unable to protect the bacteria, and the bacteria in the biofilm eventually succumb to sPBBs. Knowledge acquired when conducting kinetics and extrusion experiments will help support or disprove the mechanism of action.

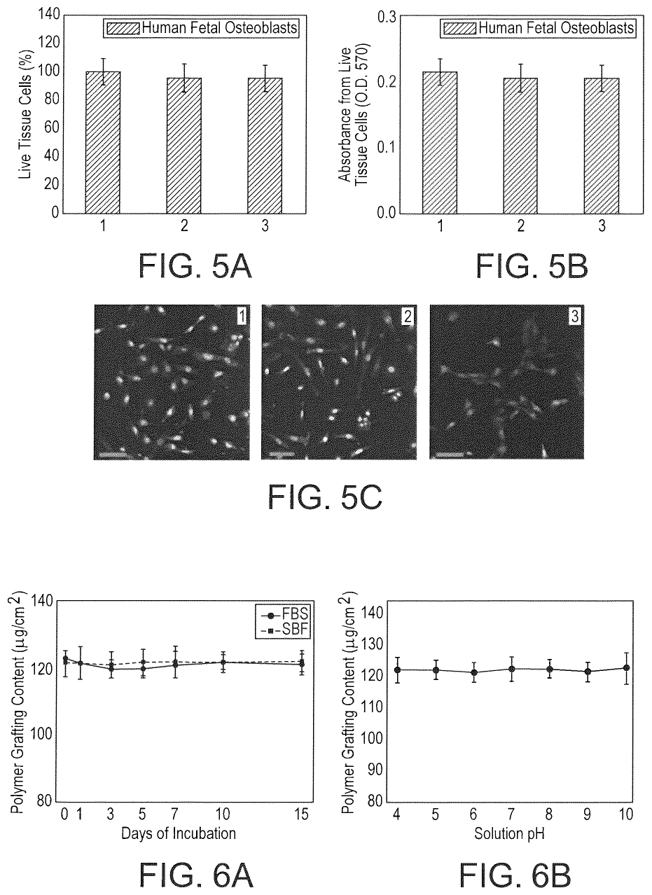

Due to the excellent anti-infective activity of sPBBs, it was imperative to test their biocompatibility using human fetal osteoblasts (HFOs), which were cultured in 6-well plates in alpha MEM medium supplemented with 10% fetal bovine serum (FBS) and incubated at 37° C. in a humidified atmosphere with 5% CO₂ for 7 days. The condition of HFOs cultured on control surfaces, as well as on sPBBs was analyzed using the MTT assay and Live/Dead staining kit (FIG. 5). Results from this study demonstrate the biocompatibility of both biomaterial surfaces and sPBBs towards HFOs (FIG. 5A). Unlike bacteria, HFOs cultured on sPBBs were healthy and grew steadily throughout the timespan of the experiment (FIG. 5A). When compared to the control surfaces, HFOs cultured on sPBBs grew similarly and demonstrated normal tissue functions and comparable cell numbers (FIG. 5B). Confocal microscopy revealed sPBBs allowed for proliferation and continuous tissue cell growth (FIG. 5C). Results from biocompatibility studies suggest sPBBs strike a balance between pathogen-killing efficacy and biocompatibility.

The lack of durability associated with old-generation surface modifications especially when in contact with blood plasma, proves to be a common problem, limiting their potential application. However, the sPBBs constructed in this study are stable over a 15-day experiment as indicated by no significant loss of polymer content demonstrated in the Orange II assay (FIG. 6). When testing the stability of sPBBs, it appears that the salts from simulated body fluid, and the proteins, enzymes, sugars, and other biomacromolecules from fetal bovine serum do not interfere with the covalent bonds between the polymer brushes and the biomaterials. In fact, the surfaces are extremely stable, allowing for less than 4 percent of polymer release even after 15 days of exposure (FIG. 6A). As demonstrated through the work of others, adherent bacteria drop the local pH of the environment when colonizing a surface.

In order to better understand the chemical effects of pH on sPBBs, polymer release in PBS solutions of various pHs were monitored (FIG. 6B). The sPBBs were stable when placed in solutions with a wide pH range for 15 days, resulting in only 3 percent of polymer brush release throughout the course of the experiment demonstrating the covalent nature of the polymer bonds.

1. An antimicrobial coating produced by a process comprising:
a) applying an inert plasma directly to a biomaterial substrate for about 1-5 minutes at a discharge power of about 60-100 watts, thereby producing an activated biomaterial,
b) exposing the activated biomaterial to air for about 50 to about 100 minutes to generate hydroperoxide, hydroxyl, and/or peroxide reactive centers on the activated biomaterial, and
c) placing the air-exposed activated biomaterial into an ammonium monomer solution with a concentration of about 20-50% (w/v) to induce graft polymerization, thereby initiating a free-radical surface grafting at reactive centers, wherein an antimicrobial biocoating is produced on the biomaterial substrate.

2. The antimicrobial coating of claim 1, wherein the biomaterial substrate comprises polyethylene terephthalate (PET), silicon wafer, cyclic olefin copolymer (COC), polycarbonate (PC). polyetherimide (PEI), medical grade polyvinylchloride (PVC), polyethersulfone (PES), polyethylene (PE), polyetheretherketone (PEEK) and/or polypropylene (PP).

3. The antimicrobial coating of claim 1, wherein the ammonium monomer is selected from the group consisting of allyltrimethyl ammonium, allyltriethyl ammonium, allyltripropyl ammonium, allyltributyl ammonium, allyltripentyl ammonium, allyltrihexyl ammonium, allyltriheptyl ammonium, allyltrioctyl ammonium, allyltrinonyl ammonium and combinations thereof.

4. The antimicrobial coating of claim 1, wherein graft polymerization occurs at about 50° C. to about 90° C. under a nitrogen atmosphere for about 6-10 hours.

5. The antimicrobial coating of claim 1, wherein the inert plasma is argon plasma.

6. The antimicrobial coating of claim 1, wherein the polymer brush length is about 270 nm to about 470 nm.

7. The antimicrobial coating of claim 1, wherein the polymer graft density is about 50 to about 200 μg/cm².

8. The antimicrobial coating of claim 1, wherein the coating inhibits the formation of biofilms.

9. The antimicrobial coating of claim 1, wherein the coating inhibits gram-positive and gram-negative bacteria.

10. The antimicrobial coating of claim 1, wherein the coating inhibits S. aureus, S. epidermidis and E. coli bacteria.

11. A method of producing an antimicrobial coating comprising:
a) applying an inert plasma directly to a biomaterial substrate for about 1-5 minutes at a discharge power of about 60-100 watts, thereby producing an activated biomaterial,
b) exposing the activated biomaterial to air for about 50 to about 100 minutes to generate hydroperoxide, hydroxyl, and/or peroxide reactive centers on the activated biomaterial, and
c) placing the air-exposed activated biomaterial into an ammonium monomer solution with a concentration of about 20-50% (w/v) to induce graft polymerization, thereby initiating a free-radical surface grafting at reactive centers, wherein an antimicrobial biocoating is produced on the biomaterial substrate.
